(19) United States
(12) Patent Application Publication
(10) Pub. No.: US 2018/0049914 A1
(43) Pub. Date: Feb. 22, 2018

(54) Thermal Composite Material and Compression Sheet

(71) Applicant: PolyPhysics, Inc., Hampton, VA (US)

(72) Inventor: Brian Keith Stewart, Hayes, VA (US)

(73) Assignee: PolyPhysics, Inc., Hampton, VA (US)

(21) Appl. No.: 15/680,327

(22) Filed: Aug. 18, 2017

Related U.S. Application Data
(60) Provisional application No. 62/377,053, filed on Aug. 19, 2016.

(51) Int. Cl. A61F 7/02

(52) U.S. Cl. CPC A61F 7/02; A61F 2007/0292; A61F 2007/0268; A61F 2007/0249

(57) ABSTRACT

A thermal composite material and devices that incorporate the material control skin temperature within a specified range during standard cold therapy or heat therapy. The material and subsequent devices can also have a structural input-output feature to apply and measure a degree of compression. A primary use is as portable means for the immediate treatment of soft tissue injury. Secondary uses include use as portable means to aid temperature regulation in hot or cold environments for comfort or safety purposes.

This application claims the benefit of U.S. Provisional Application No. 62/377,053, filed Aug. 19, 2016, entitled “Thermal Composite Material Used in Cold And/Or Heat Therapy,” which is incorporated by reference herein in its entirety.

The present invention relates to a thermal composite material and subsequent devices that incorporate this material which maintains skin temperature within a specified range during standard cold therapy or heat therapy. The material and subsequent devices can also have a structural input-output feature to apply and measure a degree of compression. One use is as portable means for the immediate treatment of soft tissue injury. Other uses include use as portable means to aid temperature regulation in hot or cold environments for comfort or safety purposes.

### BACKGROUND

The standard treatment protocol for the treatment of soft tissue injuries is rest, ice compression, elevation (R.I.C.E.). When immediately and properly applied, R.I.C.E. treatment aids recovery by reducing cell metabolism and reducing edema. This minimizes the oxygen demand in the vicinity of the injury and reduces secondary cell death of uninjured tissue resulting from hypoxia. The reduced temperatures initiate an involuntary vascular response known as vasodilation. Vasodilation increases the blood perfusion to the affected area by increasing the availability of oxygen and nutrients, and by aiding the removal of wastes and fluids. The cooling also has a local analgesic effect, reducing pain in the injured area.

Field treatment of soft tissue injury is usually performed by using ice or other cold substance enclosed in a soft bag or container. The cold substance maintains a low temperature via phase change (such as ice) or by endothermic chemical reaction. Ice and chemical cold packs create skin temperatures that are below safe levels for prolonged contact. Various means are employed to minimize the risk of cold injury including the addition of a thermal barrier (towel or other means) and the cyclic application and removal of the ice at regular intervals during long-term treatment. However, simple thermal barriers do not regulate the heat transfer, making it impossible to determine the degree of cold therapy applied. In addition, neither method provides or manages compression therapy. Elastic bandages can be applied prior to ice application, but these do not result in predictable thermal properties, making the degree of cryotherapy applied unknown. Elastic bandages provide no objective means of measuring the amount of compression applied, instead relying upon approximations by “feel’ and other subjective means of measure.

Cold therapy is a complex physiological process with multiple tissue types responding simultaneously. The purpose of cold therapy is to reduce the temperature of deeper muscle tissue; which reduces metabolism and increases blood flow by vasodilation. Compression therapy reduces edema and prevents the accumulation of waste fluids in the vicinity of the injury. The cold therapy process takes place via heat transfer through multiple layers of tissue, each with varying metabolism rates, blood perfusion rates, densities, thermal conductivity and thermal heat capacities. Heat energy is transported through the layers of tissue via temperature gradients between the deep tissue (source) and the cold substance (sink). The skin is the tissue layer where the heat is transported from the body and is subjected to the lowest (or highest) body temperatures during treatment. Deep tissue cooling requires cooling of the skin for a sustained period of time. Current field means of cooling; ice and cold packs, result in unsafe low skin temperatures, discomfort, muscle spasms and other undesirable physiological effects. Maintaining a safe skin temperature is typically managed by cyclic application and removal of the low temperature material or the inclusion of unregulated thermal barriers such as fabric layers. The result is inconsistent and unknown skin temperature during the course of long-term treatment, or temperatures that are too low for safe application during long-term treatment.

There are numerous water recirculating systems used for cold therapy that employ a water-ice mixture, a mechanical pump, and a device that is applied in contact with the skin at the injured area and connected to the pump via tubes. Cold therapy is achieved via the cold recirculating water flowing through the applied device to cool the injured area. Some versions of this type of device contain a pneumatic bladder capable of applying compression at precise levels. These devices do not regulate the temperature of the water, introducing the risk of cold injury. They often require specialized training to use safely and are not practical for field application on a large scale. In addition, they are expensive to own and operate and require qualified health professionals to conduct the therapy.

### SUMMARY

A thermal composite material is provided herein which regulates the temperature of the skin by providing a thermal regulation barrier between the skin and the cold material. The thermal composite material regulates the skin temperature within a known, safe temperature. The material also may include a function that allows measured compression therapy via elastic properties and measured via a numeric scale. Other features and advantages will become apparent upon a reading of the attached specifications, in combination with a study of the drawings.

In one example, a temperature-controlled, composite sheet for use in hot or cold therapy applications comprises a temperature control component and an elastic structural component. The temperature control component comprises a phase change material having a predetermined, therapeutic, specific temperature of phase change from solid to liquid forms of from about 32 degrees F. to about 122 degrees F. The elastic structural component is comprised of a material having elastomeric properties. The temperature control component and elastic structural component form a single, contiguous layer, or alternatively, the temperature control component is comprised of a layer of a plurality of adjacent cells. Still further alternatively, the temperature control component is comprised of a plurality of adjacent, semi-contiguous cells. The temperature control component may further comprise a matrix of elastomer material with the phase change material dispersed therein. The temperature control component may comprise an insulating material. The phase-change material may comprises about 20% to 90% by volume of the temperature control component, or alternatively about 30% to 80% by volume of the temperature control component. The elastic structural component is formed of a solid elastomer or reinforced elastomer. The sheet may further comprise a separate insulating layer. The insulating layer may have a thermal conductivity of about 0.05 to 150 W/mK. The insulating layer may be comprised of a polyurethane or silicone cell material selected from the group consisting of an open cell foam, a closed cell foam, a syntactic foam or a mixture thereof. The phase change material may have a phase change temperature below about 98.7 degrees F., or alternatively the phase change material may have a phase change temperature above about 98.7 degrees F.

In another example, a temperature-controlled, composite sheet comprises a sheet of temperature control material, wherein the temperature control material further comprises a phase change material having a predetermined, therapeutic, specific temperature of phase change from solid to liquid forms. The predetermined temperature of phase change may be from about 32 degrees F. to about 122 degrees F. The sheet may be comprised of a dispersion of the phase change material in an elastomer matrix.

### BRIEF DESCRIPTION OF THE DRAWINGS

FIG. 1 is a side, cross-sectional view of an example of a temperature-controlled, composite sheet as described herein.

FIG. 2A is a side, cross-sectional view of another example of a temperature-controlled, composite sheet as described herein.

FIG. 2B is a blown-up view of portion of the cross-sectional view as shown in FIG. 2A.

FIG. 3A is a side, cross-sectional view of a third example of a temperature-controlled, composite sheet as described herein.

FIG. 3B is a side, cross-sectional view of a fourth example of a temperature-controlled, composite sheet as described herein.

FIG. 3C is a side, cross-sectional view of a fifth example of a temperature-controlled, composite sheet as described herein.

FIG. 3D is a side, cross-sectional view of a sixth example of a temperature-controlled, composite sheet as described herein.

FIG. 4A is a top view, and a side, cross-sectional view of a seventh example of a temperature-controlled, composite sheet as described herein.

FIG. 4B is a top view, and a side, cross-sectional view of an eighth example of a temperature-controlled, composite sheet as described herein.

FIGS. 5A and 5B are top views of a ninth example of a temperature-controlled, composite sheet as described herein in an at rest (FIG. 5A) and stretched (FIG. 5B) condition.

FIG. 6A is a side, cross-sectional view of a tenth example of a temperature-controlled, composite sheet as described herein.

FIG. 6B is a top view of an eleventh example of a temperature-controlled, composite sheet as described herein.

FIG. 6C is a cross-sectional view of the composite sheet shown in FIG. 6B taken along lines 6C-6C.

FIG. 6D is a top view of a twelfth example of a temperature-controlled, composite sheet as described herein.

FIG. 6E is a partial side, cross-sectional view of the composite sheet shown in FIG. 6D taken along lines 6E-6E.

FIG. 6F is a partial side, cross-sectional view of the composite sheet shown in FIG. 6D taken along lines 6F-6F.

FIG. 7A is a side, cross-sectional view of a thirteenth example of a temperature-controlled, composite sheet as described herein.

FIG. 7B is a side, cross-sectional view of a fourteenth example of a temperature-controlled, composite sheet as described herein.

FIG. 7C is a side, cross-sectional view of a fifteenth example of a temperature-controlled, composite sheet as described herein.

FIG. 7D is a side, cross-sectional view of a sixteenth example of a temperature-controlled, composite sheet as described herein.

FIG. 8A is a top view of a seventeenth example of a temperature-controlled, composite sheet as described herein.

FIG. 8B is a side, cross-sectional view of the composite sheet shown in FIG. 8A taken along lines 8B-8B.

FIG. 8C is a side, cross-sectional view of an example of interlocking sections of the composite sheet as shown in FIGS. 8A and 8B.

FIG. 9 is a top view of an eighteenth example of a temperature-controlled, composite sheet as described herein.

FIG. 10A is a perspective view of an example of a hot water bottle or ice pack as described herein.

FIG. 10B is a side, cross-sectional view of the bottle/pack shown in FIG. 10A, taken along lines 10B-10B.

### DETAILED DESCRIPTION

A thermal composite material is presented with combinations of thermal properties that maintain a specific temperature on the skin on one side (source), while assuming the temperature of the cold temperature (or heat) source (sink) on the other. The effect is produced by controlling the values and distribution of two key thermophysical properties within the composite material—thermal conductivity and latent heat of phase change throughout the composite material via controlled quantity and dispersion of the constituent components of the composite material. Structural properties can also be distributed throughout the composite material to provide for application of, and precise measurement of. applied compression.

This material can be used for controlling heat removal or heat addition from the skin and underlying tissue. For simplicity, descriptions herein address the function mostly in terms of cooling alone (heat removal), with the reverse function of heating tissue (heat addition) at a controlled skin temperature intended throughout.

The composite material consists of multiple functional domains that may also be referred to and embodied herein as layers; a Temperature Control domain, a Structural Control domain and, optionally, an Insulation domain. The functions of these domains/layers can be distinct (separate layers for each functional domain), or they can be combined in a single physical layer. The specific geometric shape of each domain/layer can vary depending upon the specific application. For example, the respective functional domains could be arranged in distinct layers, although numerous other arrangements are possible. Sometimes, the functional domains may be incorporated into one or more combined layers.

### Temperature Control Domain

The temperature control region of the composite material provides the primary mechanism for skin temperature control functionality. The temperature control is achieved by encapsulation of a phase change material (PCM) into a soft matrix component. PCM's change from solid-liquid or liquid-vapor at or near a specific, known temperature. The chemistry of the different PCM's will control the phase change temperature of each PCM. Phase change temperatures are important, because a significant amount of thermal energy is required to transform from one phase to another. Therefore, a thermal energy barrier is created to absorb and release large heat fluxes at temperatures near the phase change temperature. As long as the heat source (tissue) produces heat energy, and the heat sink (cold source) maintains a sufficiently low temperature, the phase change material will always have a region where two phases co-exist. This phase interface region will be at the material phase change temperature (melting point or boiling point) of the PCM. By placing the Temperature Control domain in contact with the skin, the skin temperature will be maintained at some temperature near the PCM phase change temperature, thus providing precision skin temperature control with a passive system. The energy potential driving the process is the temperature difference between the source and the sink, with heat flow regulated by the phase change material phase of the composite.

For the purposes of the present invention, the phrase “phase change material” shall mean a material having a specific, predetermined phase change temperature in the range of between about 32 degrees F. (zero degrees C.) and about 122 degrees F. (50 degrees C.). Typical PCM's may include various fatty acids, paraffins, inorganic phase change compounds and similar substances that transform from solid-liquid phase in the range of temperatures noted above. Suitable fatty acid PCM's include, but are not limited to, the following: linoleic acid, oleic acid, stearic acid, palmitic acid, and lauric acid. Suitable paraffin PCM's include, but are not limited to the following: n-Tetradecane (C14), n-Hexadecane (C16), n-Octadecane (C18), n-Eicosane (C20), n-Octadecane (C22), and n-Tetracosane (C24). Suitable inorganic PCM's include, but are not limited to, Sodium sulfate decahydrate and Calcium chloride hexahydrate. Moreover, the PCM's incorporated into a composite material may be a mixture of different PCM compounds in order to achieve a targeted phase change temperature or range of temperatures.

The phase change material may be incorporated into the composite structure at a high volume percentage or as a generally pure compound or mixture of compounds. When in a high volume percentage or generally pure form, the material may be relatively fluid or viscous. Therefore, it is typically inserted into cells that are defined by an impermeable, thin barrier film layer that defines the outside of the cell.

Alternatively, the PCM's may be incorporated into the composite structure as an encapsulated compound or mixture of compounds. These encapsulated particles are dispersed within a polymer matrix of an elastomeric polymer including, but not limited to, a silicone material. These encapsulated PCM's are often referred to with reference to their size as “macro” or “micro” encapsulated particles. In each case, the PCM material is encapsulated in a thin outer layer of a more rigid polymer such as, for instance, polyethylene. “Macroencapsulated PCM” describes the example where the phase change material particles have a size of about 100 to 6000 microns in diameter, or alternatively about 200 to 3000 microns. The particle shape may be generally spherical, and are referred to in size as measuring diameter, but they may be other shapes as well. “Microencapsulated PCM” describes the case where the PCM encapsulation dimension is much smaller, for example about one to a hundred microns in diameter. Again, the shape is referred to as spherical, but the shape will have some variations.

In addition to thermal properties imparted by the PCM that is used, the Temperature Control domain may have structural functionality. This structural behavior can be combined with that of a separate, independent Structural domain. Alternatively, the polymer matrix that has a PCM mixed or dispersed therein may have a desirable, or may be engineered to have a desirable structural functionality to where the composite material can serve in an intended application. The temperature control functionality can also be combined with an insulation functionality by the additional inclusion of constituent materials with insulating properties into a Temperature Control domain composite mixture.

In one example, it is this temperature control functionality that is deployed in a layer of the composite sheet that is positioned next to the skin of a user. This temperature control layer, through the inclusion of PCM's in one or more sealed cells that make up this layer or through PCM's that are mixed or dispersed within this layer, imparts the beneficial functionality that prevents a person's skin from becoming damaged or injured as a result of too much cold or heat therapy. Therefore, regardless of whatever else is dispersed within or alternatively placed into the sealed cells of the temperature control layer, there must be incorporated some amount of PCM in this layer to receive the therapeutic benefit of temperature control.

The amount of PCM that is used in a temperature control domain will depend on the physical makeup of the chosen domain. In one example, a pure or nearly pure PCM is loaded into a sealed cell or multiple cells that is/are sealed on a structural substrate or backing sheet. A thin barrier film, in one example a polyethylene film, defines the cell and holds the PCM therein. The filler material is about 100% PCM , or alternatively about 75-100% PCM. Other filler materials may be added as noted earlier including insulating materials and/or other additive components to improve flowability or dispersion of the mixture. In another example, the PCM is mixed/dispersed within a polymer matrix to form a generally homogeneous composite material. The polymer matrix can be, in one example, a rubber or silicone or other elastomer compound. The PCM material constitutes about 20% to 90% by volume of this composite material, or alternatively about 30% to 80% by volume of this composite material, or in one example about 40% of the composite material by volume.

The particular PCM that is chosen for use may have a phase change temperature either at, below, or above about 98.7 degrees F., a common human body temperature. A PCM phase change temperature below body temperature is used to modulate the cooling of the skin of injured body part. As explained earlier, the temperature of 32 degrees F. (ice/water melting/freezing point) may damage or injure a person if applied too long. Accordingly, PCM phase change temperatures between about 35 and 65 degrees F., or alternatively about 40 and 50 degrees F. may be preselected to deliver a therapeutic amount of cooling treatment as determined by a doctor or therapist or trainer. On the heating side of therapy, the PCM phase change temperature is above about 98.7 degrees F., alternatively about 100 to 120 degrees F., or still further alternatively, about 105 to 115 degrees F. Finally, it is also possible that the desirable phase change temperature is at or close to body temperature—about 98.7 degrees F.

### Structural Domain

The structural domain provides for structural integrity and connectivity of the composite material; independently or combined with that of the Temperature Control domain. In practice, this structural domain is a sheet layer that has the temperature control layer attached onto or integrated into the sheet. The structural sheet may be square or rectangular, or asymmetric in shape, flat or curved to fit a particular anatomic application. In a common generic application, the structural layer is in the form of a tape having a width of about 2 to 12 inches, or alternatively about 3 to 6 inches. Another generic application would be a rectangular or round bag shape to mimic a classic ice bag or hot water bag. A third application would be a curved or contoured shape designed to fit a specific body part or region.

In order to control the structural contribution from the Temperature Control domain, the Temperature Control domain can be divided into non-contiguous segments, a contiguous layer, or some combination of contiguous and non-contiguous features.

The Structural domain or sheet layer consists of either a solid elastomer or mixture of elastomers, reinforced elastomer, or foamed elastomer . It can function as a means of mere connectivity to render a structurally cohesive material, or it may serve as a means of providing compression to the affected area by virtue of engineered elastic properties. The fiber reinforcement can be tailored to provide precise force-displacement relationships to yield specific compression performance, to increase the stiffness, or to improve durability.

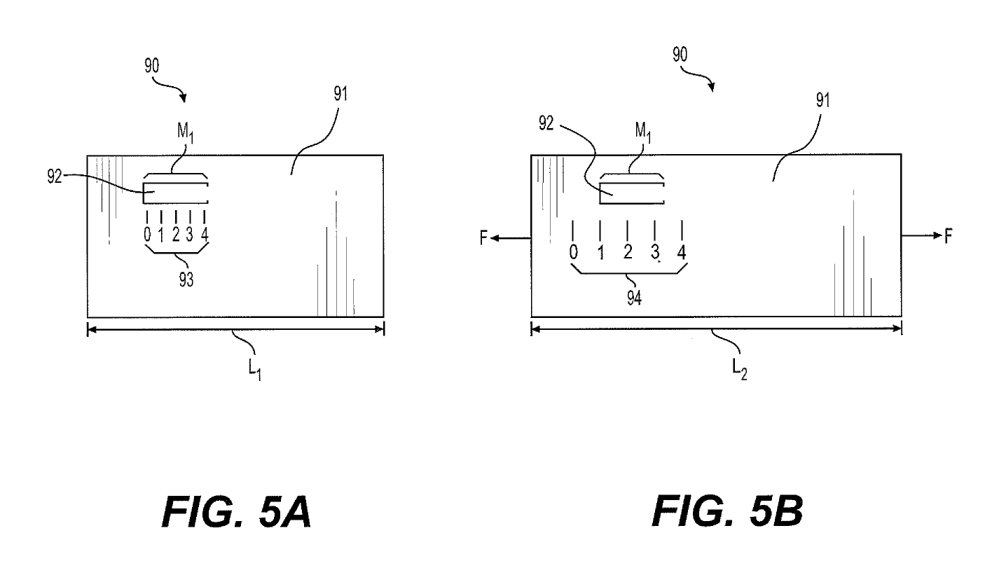

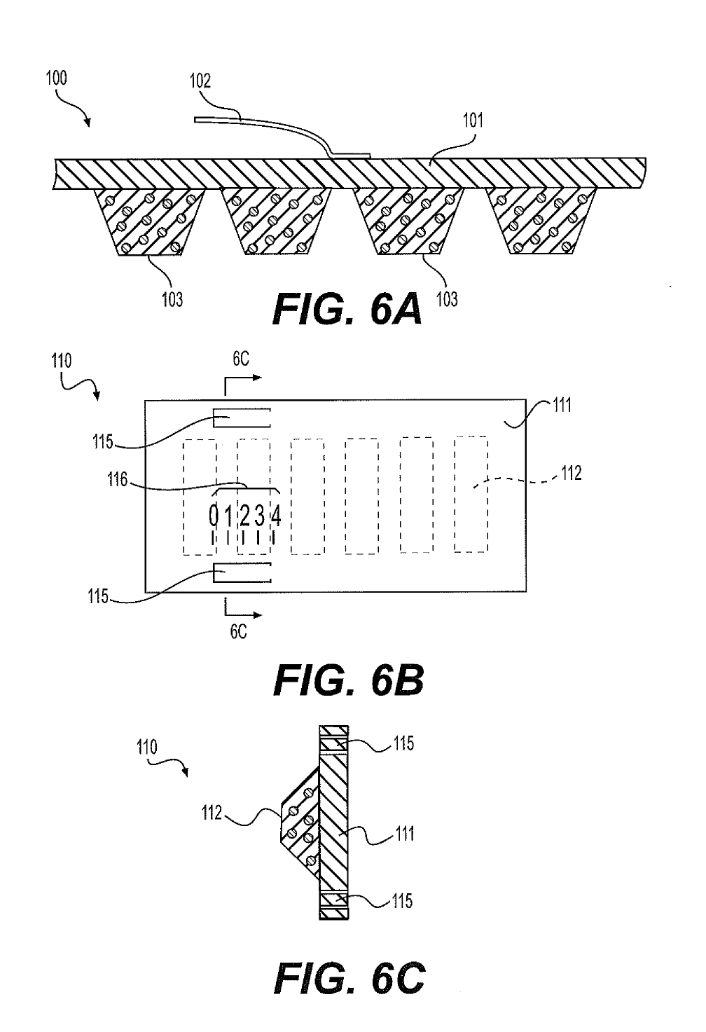

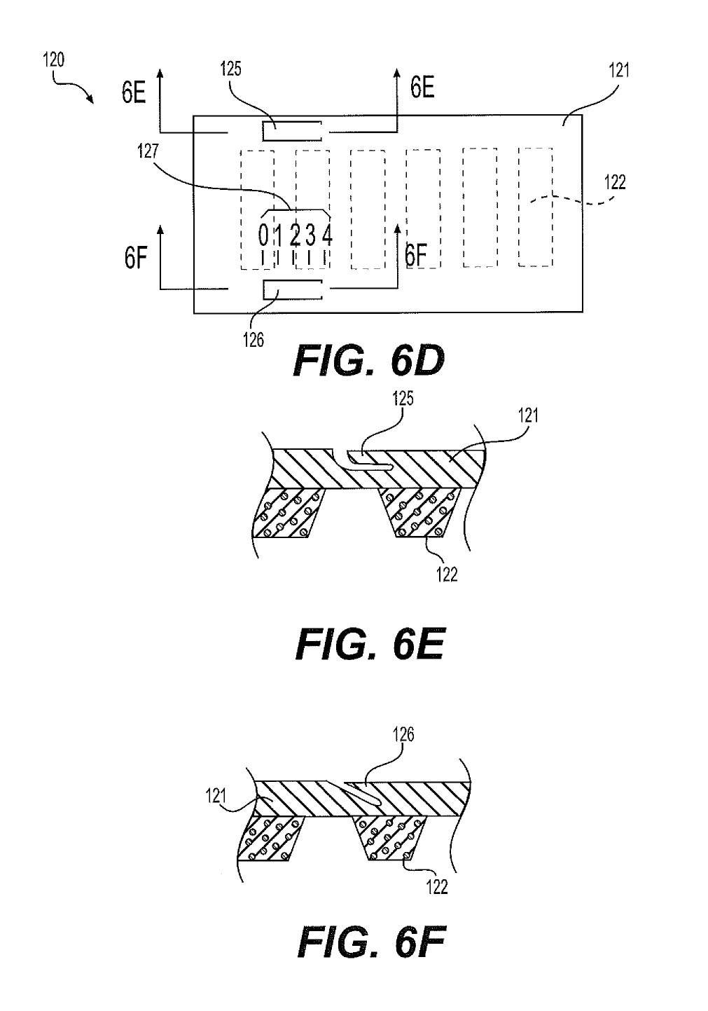

The addition of a non-elongating feature (tab) and a visual indicia of a scale on the elastic, structural sheet can provide a means of measuring the elongation of the structural sheet, and thus provide a ready means of visually measuring the applied compression. One embodiment of this is shown in FIGS. 5A and FIG. 5B for a thin elastic sheet with a tab attached at one end. When the structural layer is elongated, the tab length is unchanged. The edge of the tab aligns with marks on the elongating structural domain, providing a means of measuring the elongation. The relationship between stretch and the force applied provides a repeatable means of measuring the tension, and thus the degree of compression applied during use. Alternate embodiments of incorporating a tab feature are shown in FIGS. 6A-6F.

### Insulation Domain

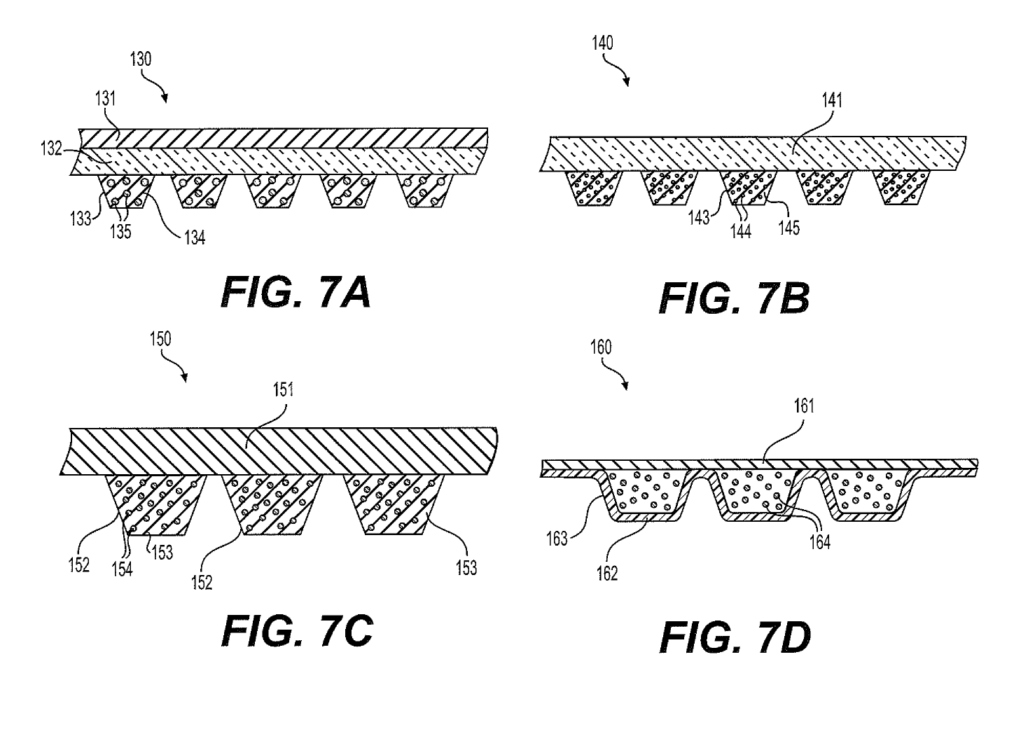

The insulation domain is the primary means to control the thermal conductivity of the composite. Both the Temperature Control and Structural Control domains have finite thermal conductivities that will impede heat transfer to some degree. The purpose of the incorporation of an insulating material is to provide additional, deterministic control over heat transfer independent of the other domains. FIG. 7A shows an insulation layer using low conductivity hollow microspheres dispersed into a soft polymer matrix as a distinct layer. FIG. 7B shows similar microspheres dispersed into a structural layer, while FIG. 7C shows microspheres dispersed into the material that constitutes the cells that make up the Temperature Control domain in combination with the PCM microspheres. FIG. 7D shows low conductivity microspheres dispersed in a macro encapsulated PCM. The low conductivity constituent can take the form of hollow microspheres. Alternative embodiments include low conductivity constituents in the form of solid particles, short fibers, syntactic foams, closed-cell foams or open-cell foams.

### Self-Attachment Features

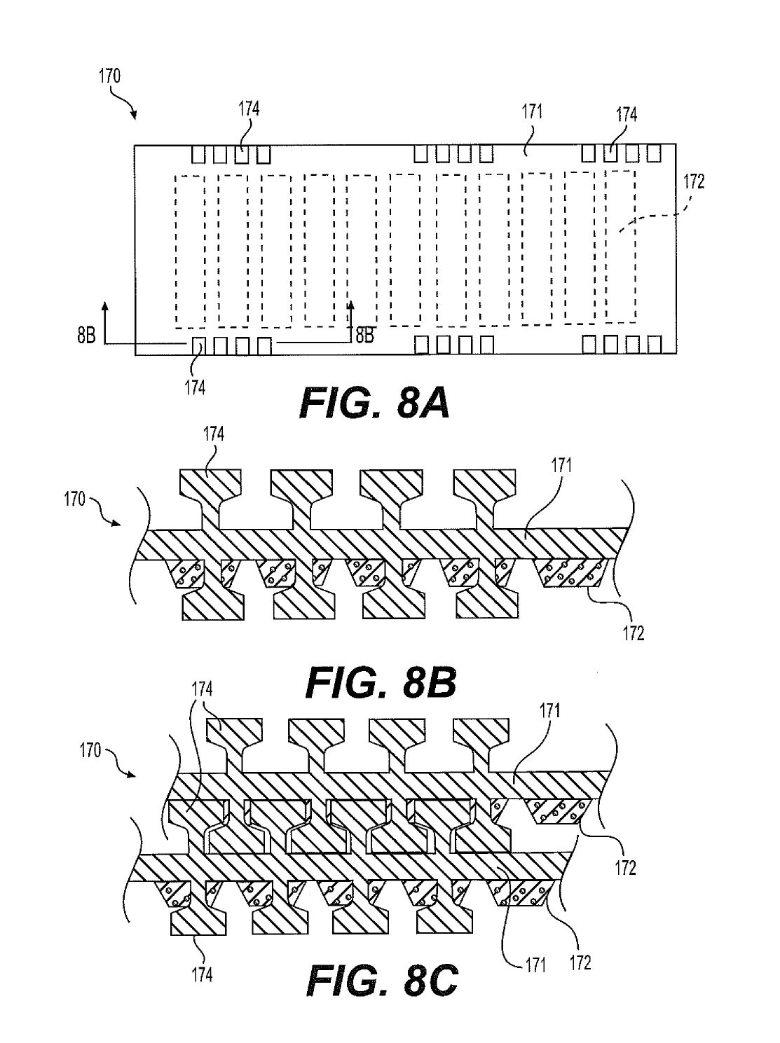

To provide a means of securing the temperature control sheet in-situ, a series of locking protuberances can be included on portions of both sides of the structural domain layer. The protuberances interlock with each other, after application to (and possibly stretching over) the injured area. This feature secures the composite material independent of external means such as tape, clamps or stretch wrap. The protuberances plan view, and sections showing unfastened and fastened protuberances are shown in FIGS. 8A-8C.

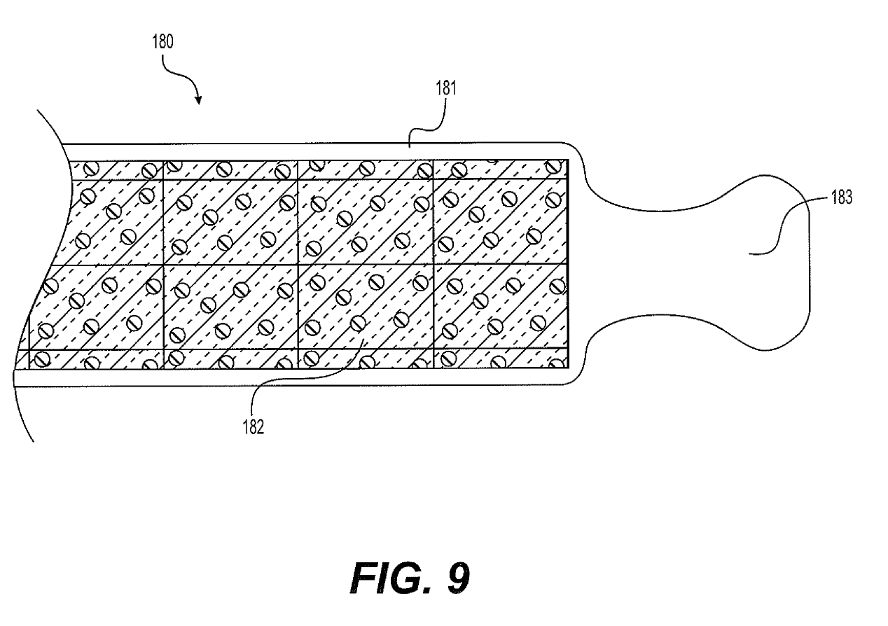

For a tape form embodiment, the material can be secured by wrapping and tucking the end of the tape under the wrapped portion. To accommodate this feature, extended tabs can be included at the ends of the tape (FIG. 9) to facilitate fastening by this method.

The composite sheet has been discussed generally so far. Reference to multiple examples of the composite sheet is now made in the context of the drawings.

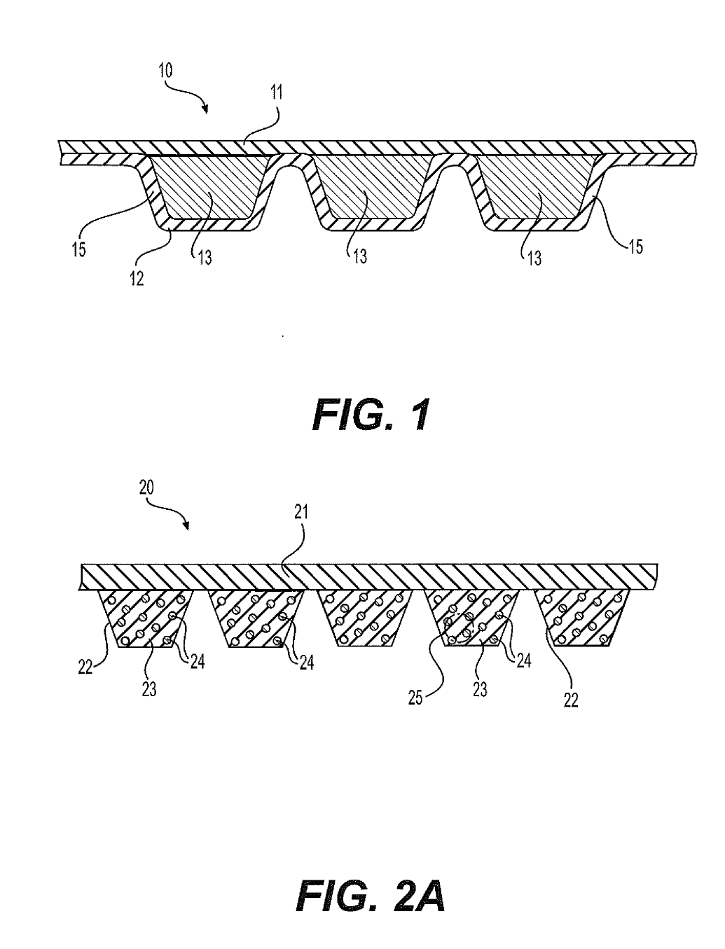

FIG. 1 is a side, cross-sectional view of a composite sheet 10. The composite sheet 10 has a silicone backing layer 11. A barrier film 12 forms the cells 15 that are filled with phase change material 13 in the form of one or more paraffins. The composite sheet 10 as a whole is flexible and stretchable because of the nature of the formative components described above. And while the material 13 filled into the cells 15 defined by the barrier film 12 is essentially 100% PCM, there could be less PCM by volume with additional filler items added for desired functional properties.

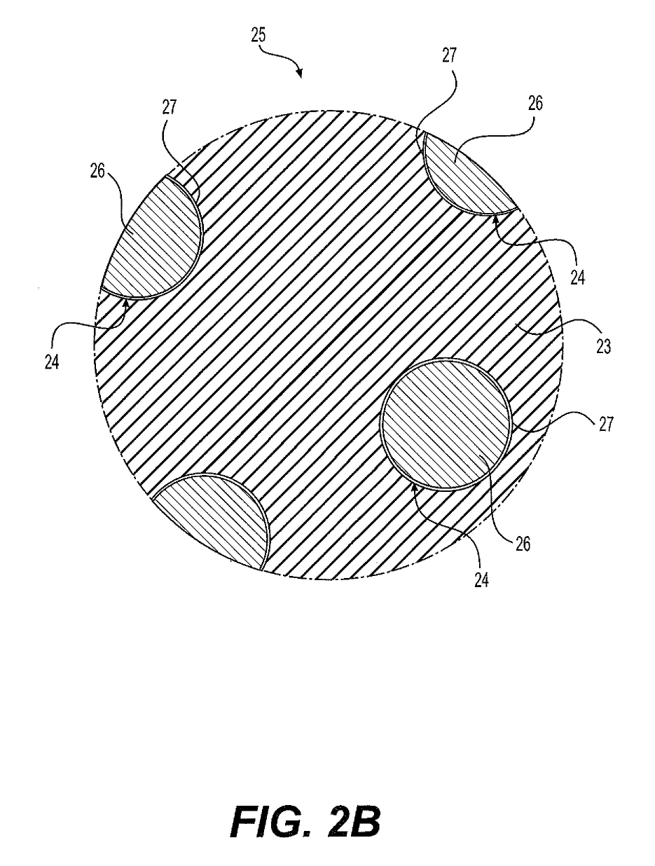

FIG. 2A is a side, cross-sectional view of composite sheet 20. Composite sheet 20 has a flexible backing sheet layer 21 formed of silicone. Attached to the backing sheet layer 21 are cells 22 formed of encapsulated PCM 24 particles dispersed in a silicone matrix 23. FIG. 2B is a close-up view of a portion 25 of the cell 22. The silicone matrix 23 has the encapsulated PCM 24 dispersed in it. The encapsulated PCM 24 is shown spherical in shape. The encapsulated PCM 24 has an outer shell 27 that seals inside the PCM 26. The outer shell 27 is a polyethylene or other polymer that protects and retains the PCM 26 inside. While the encapsulated PCM 24 is believed to be generally spherical in shape, obviously there are expected variations in shape that may be present. These capsules are referred to as spherical and capture these other variations in shape by definition.

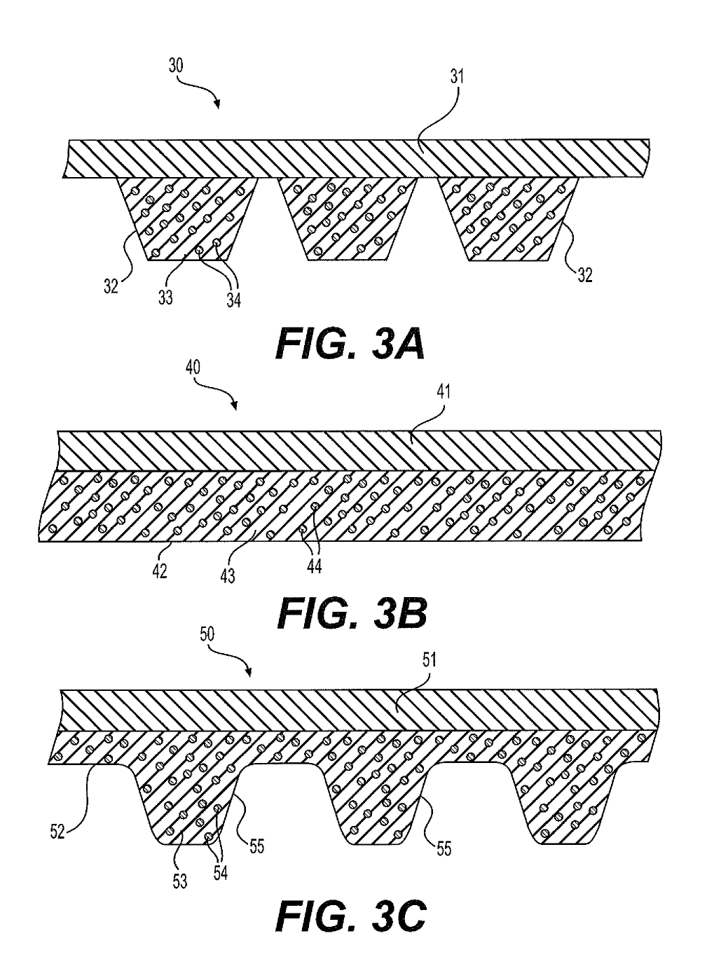

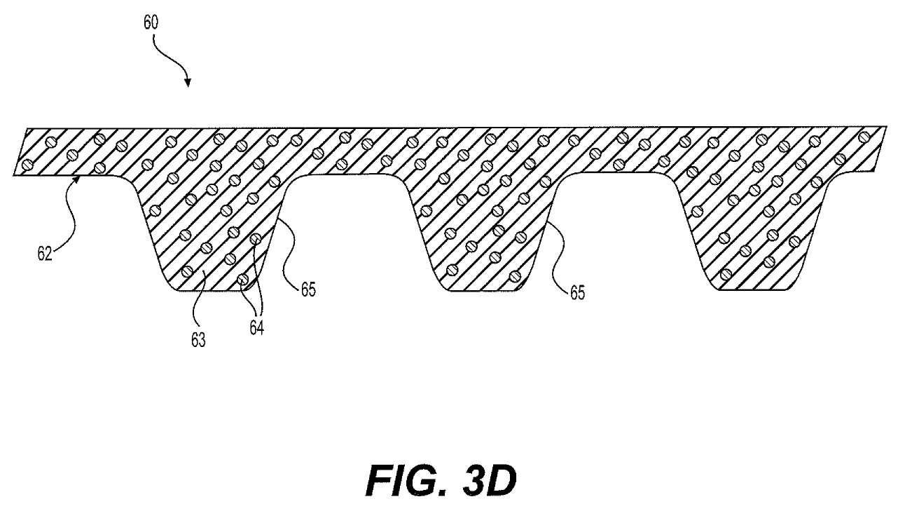

FIGS. 3A-3D illustrate alternative physical examples of a flexible composite sheet. In FIG. 3A, the composite sheet 30 has a silicone backing layer 31 and cells 32 attached on one side of the backing layer. The cells 32 are formed of a silicone matrix 33 with encapsulated PCM 34 dispersed therein. The cells 32 are noncontiguous on the silicone backing layer 31.

FIG. 3B displays a composite sheet 40 having a flexible silicone backing layer 41. On one side of the silicone backing layer 41 there is a generally uniform layer of temperature control material 42 that is formed of encapsulated PCM 44 dispersed in a silicone matrix 43.

FIG. 3C is a composite sheet 50 having a silicone backing layer 51. On one side thereof, there is a temperature control material 52 having cells 55 spaced therein. The material 52 and cells 55 are formed of encapsulated PCM 54 in a silicone matrix 53. This temperature control material 52 is referred to as a semi-contiguous layer.

FIG. 3D is a composite sheet 60 formed of a temperature control material 62 with cells 65 formed therein. The material is formed of encapsulated PCM 64 dispersed in a silicone matrix 63. In this example, there is no separate backing layer. The functionality of the composite sheet 60 is all in the single temperature control material layer 62.

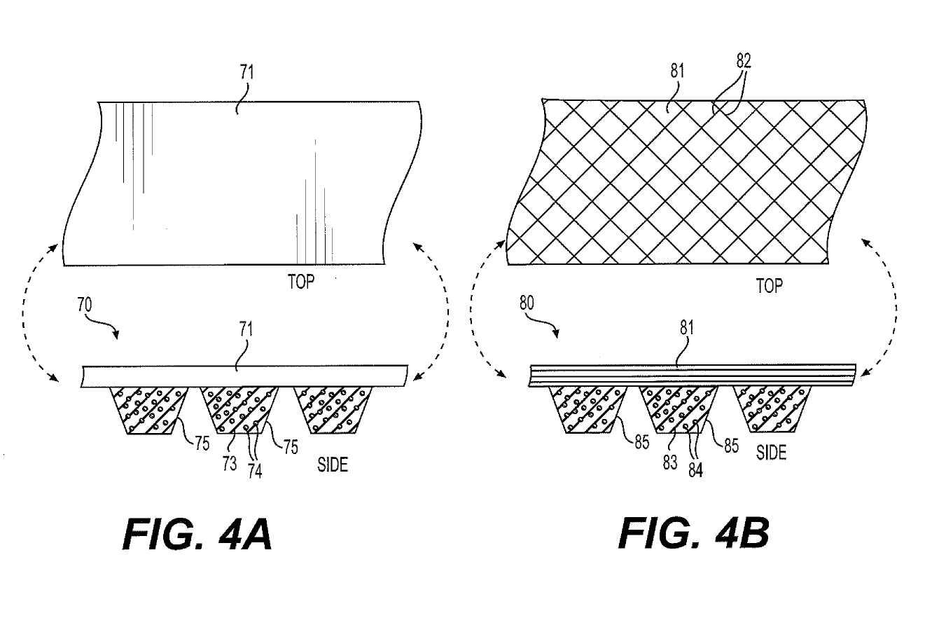

FIGS. 4A and 4B illustrate composite sheets 70 and 80. Each composite sheet 70 and 80 have comparable cells 75 and 85 formed of temperature control material made of encapsulated PCM 74 and 84 dispersed in a silicone matrix 73 and 83 respectively. The difference between the composite sheets 70 and 80 is in the backing layers 71 and 81. Backing layer 71 is formed of essentially only silicone or other elastomer. Backing layer 81 is formed of silicone/elastomer and reinforcing fibers 82. The reinforcing fiber 82 may be uniformly woven, nonwoven, randomly dispersed in, and may be made of long or short fibers or both.

FIGS. 5A, 5B, and 6A-6F illustrate different ways that the amount of compression of an applied composite sheet can be measured. As explained herein, these composite sheets may be wrapped around an arm or leg or ankle or wrist or other body part to regulate the temperature imparted to the skin. In view of the elastomeric nature of the materials used, compression may also be applied by stretching the composite sheet when applied. In order to measure or repeat a certain amount of compression, some measurement tool is needed. These figures show several ways where the amount of compression, as measured by the amount of stretch of the sheet, is applied. FIG. 5A displays a composite sheet 90 formed of an elastomer sheet 91 having a length L1. Written indicia 93 is printed on the surface of the sheet 91 adjacent a non-extendible tab 92 having a length Ml. The composite sheet 90 in FIG. 5A is shown at rest with no stretching forces. In FIG. 5B, there is shown the same sheet 90 with lateral stretching forces F applied that extends the sheet 90 to length L2 which is longer than L1. The written indicia 94 are stretched/extended accordingly, however, the non-extendible tab 92 remains the same length M1 as before in FIG. 5A. In FIG. 5A, the tab 92 is adjacent number “0” of the written indicia 93, while in FIG. 5B, the tab 92 extends to number “1” in the stretched indicia 94. Using this reference method, the amount of stretching, and consequent amount of compression can be accurately repeated.

FIG. 6A illustrates a composite sheet 100 with a flexible backing layer 101 and temperature control cells 103 attached on one side of the backing layer. On the opposite side of the backing layer 101 where the cells 103 are attached, a rigid tab 102 is attached. The rigid tab 102 may be compared with measurable indicia (not shown) on the face of the backing layer 101 that is stretched during application. The tab 102 is therefore the measuring means to establish visually a repeatable amount of compression.

FIGS. 6B and 6C are a top and cross-sectional views of a composite sheet 110. A flexible backing layer 111 of silicone has temperature control cells 112 positioned on one side thereof. On the opposite side thereof there are written indicia 116. Adjacent the written indicia 116 are tabs 115 that are cut tabs of the silicone backing layer. The physical result of the cut tabs 115 means that those tabs are not subject to any stretching of the sheet 111 overall during application. Therefore, the tabs 115 may be used as references as compared to the indicia 116 when the sheet 111 is stretched during use.

FIGS. 6D, 6E and 6F are alternative measurement methods where the backing sheet 121 can be cut to for measurement tabs. The composite sheet 120 is formed of a flexible elastic backing layer 121. Temperature control cells 122 are positioned on one side of the backing layer 121. Written indicia 127 are printed onto the surface of the backing layer 121. On the same side of the backing layer 121 as the indicia 127, and opposite the cells 122, there are tabs 125 and 126 that are cut a part of the way through the backing layer. As shown in FIG. 6E, the cut can be a uniform tab 125. As shown in FIG. 6F, the cut can be a scarf cut to for tab 126. In either example, the tab 125 or 126 will not stretch when the rest of the sheet 121 is stretched during use. The unchanged length of the tabs 125 or 126 may be compared with the stretched indicia 127 to quantify an amount of applied compression in use.

FIGS. 7A to 7D are alternative examples of ways that an insulating material may be incorporated into the composite sheets described herein. In FIG. 7A there is shown a multi-layer composite sheet 130. The composite sheet 130 includes a silicone backing layer 131 adjacent an insulating layer 132 that is mounted between the backing layer and temperature control cells 133. The cells are formed of encapsulated PCM 135 dispersed in a silicone matrix 134.

In FIG. 7B, a composite sheet 140 has a backing layer 141 that includes both elastomeric silicone and insulating material mixed together. Temperature control cells 143 are attached to one side of the combination backing layer 141. The cells 143 are formed of encapsulated PCM 144 dispersed in a silicone matrix 145.

In FIG. 7C, a composite sheet 150 has a silicone backing sheet 151 with temperature control cells 152 positioned thereon. The cells 152 in this example are comprised of encapsulated PCM 154 in a matrix of both silicone and insulating material 153. In other words, an insulating functionality is mixed into the cells 152.

FIG. 7D illustrates a composite sheet 160 having a silicone backing layer 161 and sealed temperature control cells 163 mounted on one side thereof. The cells 163 are defined by a barrier film 162 that seals a mixture of encapsulated PCM 164 and insulating material therein.

FIGS. 8A to 8C illustrate interlocking knobs that are one method of securing the composite sheet to itself during use. While similar to hook and loop fasteners, which may also be used, these interlocking knobs are flexible and formed of silicone like much of the composite sheet. In FIG. 7A, the composite sheet 170 is formed of a silicone backing sheet 171 with temperature control cells 172 positioned on one side thereof. The backing layer 171 also has adjacent knobs 174 formed therein as best seen in side view FIG. 8B. When wrapped around over itself, the composite sheet 170 is aligned so that the knobs 174 overlap. With simple pressure, the knobs 174 will interlock as shown in FIG. 8C. In this way, the sheet 170 can be removably secured to itself in use.

FIG. 9 illustrates a simple mechanical way to secure a sheet when wrapping over itself. The composite sheet 180 includes a silicone backing layer 181 and a temperature control layer 182. On one end of the sheet 180, in this view embodied as a tape, there is a tab 183. This tab portion is simply tucked under an earlier layer of sheet 180 in order to secure the tape in a removable fashion.

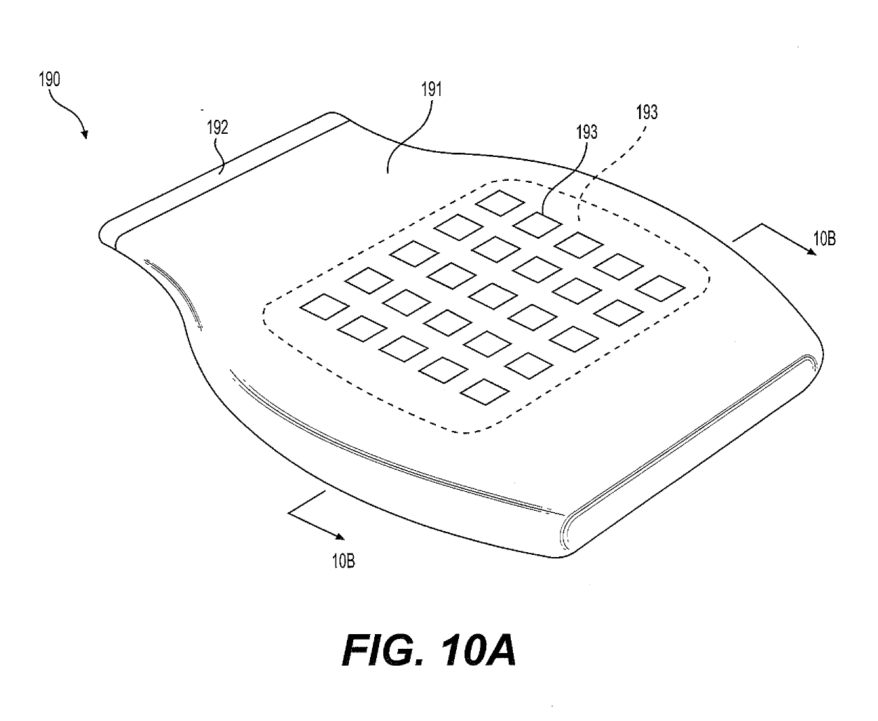

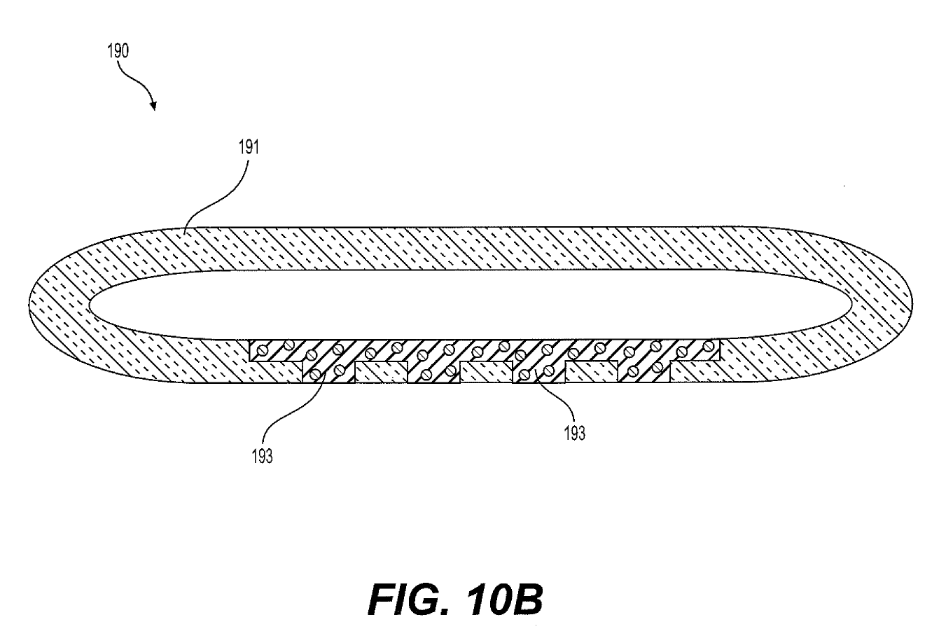

FIGS. 10A and 10B illustrate a hot water bag or ice bag alternative of the present description. The cold/hot bag 190 includes a bag layer 191 comprised of a flexible material, for instance a solid or insulating silicone, and an opening 192 where a cap or other seal may be applied. The bag 190 includes a temperature control section 193 that comprises a temperature control material including a phase change material incorporated therein.

Several examples of temperature-controlled compression sheets will now be described in the following.

### EXAMPLE 1

A tape form-factor measures 3 inches wide, 36 inches long and a total of 0.15 inches thick. The Structural domain consists of a 0.090 inch thick layer of Ecoflex 00-30® platinum cure silicone (Smooth-On, Inc, Macungie, Pa.) with PMS 3292C green colorant (Smooth-On, Inc, Macungie, Pa.) by volume added for color . The Structural domain layer is a contiguous film and measures 3 inches across and 24 inches long.

The Thermal Control domain is a mixture of Ecoflex 00-30® platinum cure silicone with 40% (by volume) microencapsulated PCM MPCM18D (Microtek, Inc., Dayton Ohio) dispersed throughout. The microencapsulated PCM is approximately spherical with an average particle size of 17 microns. The engineered melting point for the PCM is 64 degrees F. (18 deg C.). The Thermal control domain structural contribution is minimized by casting as numerous separate cell segments along the length of the material-consistent with the non-contiguous cell segments illustrated in FIG. 3A. In this particular embodiment, the discrete non-contiguous regions are identical serpentine shapes approximately 0.25 inches wide and 2.5 inches long and 0.09 inches thick. There is no colorant added, and the white color is a result of the white translucent color of the silicone and number of the microencapsulated PCM particles. This embodiment may or may not include a self-attachment feature.

### EXAMPLE 2

This embodiment is identical to Example 1, except that a 0.06″ thick Insulation domain layer is added between the Thermal Control and Structural domain. The Insulation domain consists of Ecoflex 00-30® platinum cure silicone (Smooth-On, Inc, Macungie, Pa.) with 40% by volume #22 hollow glass microspheres (Fiberglast Developments Corp., Brookfield, Ohio). Average particle size is less than 70 microns. The Insulation layer is contiguous, and thus has a nascent structural contribution in combination with the Structural domain. This embodiment may or may not include a self-attachment feature.

### EXAMPLE 3

This embodiment is identical to Example 1, with the exception that the Thermal Control domain is contiguous in the form of a single cell. It therefore has nascent structural value that combines with the structural performance of the Structural domain. This embodiment may or may not include the self-attachment feature.

### EXAMPLE 4

This embodiment is identical to Example embodiment 1 with the exception that the Thermal Control domain consists of a regular or irregular pattern of small cell shapes, approximately 0.25 inches across and distributed uniformly across the tape. This embodiment may or may not include the self-attachment feature.

### EXAMPLE 5

This embodiment is identical to Example embodiment 1 except that a series of tabs are cut into the Structural domain, near the edges so not to impact the Thermal control domain. The tabs are cut through in a manner consistent with FIG. 6B, are approximately 0.125 inches wide and 0.50 inches long. A scale is screen-printed. This embodiment may or may not include the self-attachment feature.

### EXAMPLE 6

This embodiment shows an enclosed containment where the thermal control domain extends through the entire thickness of the containment. A foam (syntactic, open-cell or closed-cell foam is cast around the thermal control domain (FIG. 10). Such an arrangement is suitable for an ice pack or similar device where the insulating layer prevents condensation and extends treatment duration, and the thermal control domain controls skin temperature.

Other embodiments of the present invention will be apparent to those skilled in the art from consideration of the specification. It is intended that the specification and figures be considered as exemplary only, with a true scope and spirit of the invention being indicated by the claims.

That which is claimed is:

1. A temperature-controlled, compression sheet for use in hot or cold therapy applications, the sheet comprising:
a composite sheet comprising a temperature control component and an elastic structural component;
the temperature control component comprises a phase change material having a predetermined, therapeutic, specific temperature of phase change from solid to liquid forms in the range of from about 32 degrees F. to about 122 degrees F.; and
the elastic structural component is comprised of a material having elastomeric properties.

2. A temperature-controlled, compression sheet as described in claim 1, wherein the temperature control component and elastic structural component form a single, contiguous layer.

3. A temperature-controlled, compression sheet as described in claim 1, wherein the temperature control component is comprised of a layer of a plurality of adjacent cells.

4. A temperature-controlled, compression sheet as described in claim 1, wherein the temperature control component is comprised of a layer of a plurality of adjacent, semi-contiguous cells.

5. A temperature-controlled, compression sheet as described in claim 1, wherein the temperature control component further comprises a matrix of elastomer material with the PCM material dispersed therein.

6. A temperature-controlled, compression sheet as described in claim 5, wherein the temperature control component is a mixture that further comprises an insulating material.

7. A temperature-controlled, compression sheet as described in claim 1, wherein the phase-change material comprises about 20 to 90% by volume of the temperature control component.

8. A temperature-controlled, compression sheet as described in claim 1, wherein the phase-change material comprises about 30 to 80% by volume of the temperature control component.

9. A temperature-controlled, compression sheet as described in claim 1, wherein the elastic structural component is formed of a solid elastomer.

10. A temperature-controlled, compression sheet as described in claim 1, wherein the elastic structural component is formed of a reinforced elastomer.

11. A temperature-controlled, compression sheet as described in claim 1, further comprising a separate insulating layer.

12. A temperature-controlled, compression sheet as described in claim 11, wherein the temperature control component is a separate layer, the elastic structural component is a separate layer, and the insulating layer is a separate layer.

13. A temperature-controlled, compression sheet as described in claim 11, wherein the temperature control component and elastic structural component form a single layer, and the insulating layer is a separate layer.

14. A temperature-controlled, compression sheet as described in claim 11, wherein the insulating layer has a thermal conductivity of about 0.05 to 150 W/mK.

15. A temperature-controlled, compression sheet as described in claim 11, wherein the insulating layer is comprised of a polyurethane or silicone cellular material selected from the group consisting of an open cell foam, a closed cell foam, syntactic foam and a mixture thereof.

16. A temperature-controlled, compression sheet as described in claim 1, wherein the phase change material has a phase change temperature below about 98.7 degrees F.

17. A temperature-controlled, compression sheet as described in claim 1, wherein the phase change material has a phase change temperature above about 98.7 degrees F.

18. A temperature-controlled, composite sheet comprising:
a sheet of temperature control material,
wherein the temperature control material further comprises a phase change material having a predetermined, therapeutic, specific temperature of phase change from solid to liquid forms.

19. A temperature-controlled, composite sheet as described in claim 18,
wherein predetermined temperature of phase change is in the range of from about 32 degrees F. to about 122 degrees F.

20. A temperature-controlled, composite sheet as described in claim 19,
wherein the sheet is comprised of a dispersion of the phase change material in an elastomer matrix.
